(19) United States
(12) Patent Application Publication
(10) Pub. No.: US 2024/0261701 A1
(43) Pub. Date: Aug. 8, 2024

(54) EXTRACTION METHODS AND APPARATUS

(71) Applicant: PUREPLANTE BIOSCIENCE INC., Bolton (CA)

(72) Inventors: Jie QI, Toronto (CA); Harry LIU, Arlington, VA (US)

(21) Appl. No.: 18/564,676

(22) PCT Filed: May 27, 2022
(86) PCT No.: PCT/CA2022/050863

§ 371 Date: Nov. 28, 2023

Related U.S. Application Data
(60) Provisional application No. 63/194,349, filed on May 28, 2021.

(51) Int. Cl. B01D 11/02

(52) U.S. Cl. CPC B01D 11/0265

(57) ABSTRACT

Methods for extracting one or more compounds from a plant or fungal material feedstock include immersing the plant or fungal material feedstock in a liquid, subjecting the immersed plant or fungal material and/or the liquid to ultrasonic vibrations, and subsequently extracting one or more compounds from the plant or fungal material. Also disclosed are methods for treating one or more compounds extracted from a plant or fungal material feedstock. Such methods include providing the extracted compounds and a liquid to an emulsification vessel, subjecting the extracted compounds and the liquid to ultrasonic vibrations using a circulating agitator to create an emulsion of the extracted compounds and the liquid. The methods for extracting one or more compounds from a plant or fungal material feedstock and the methods for treating one or more compounds extracted from a plant or fungal material feedstock may be used separately or in conjunction with each other.

### CROSS-REFERENCE TO RELATED APPLICATION

This disclosure claims the benefit of U.S. provisional application No. 63/194,349, filed May 28, 2021, the contents of which are incorporated herein by reference in their entirety.

### FIELD

This disclosure relates generally to methods and apparatus for extracting compounds from a plant or fungal material feedstock, and more specifically to methods that include pre-treatment of the plant or fungal material prior to extraction, and/or post-extraction treatment of the extracts.

### INTRODUCTION

Compounds extracted from plant and fungal material (which may be characterized as botanical extracts) have been becoming increasingly desirable in the health and wellness industry (e.g. vitamins, supplements) and cosmetics industry, among others. In particular, cannabis extracts have become more broadly available.

There are a number of known methods to extract various compounds from plant and fungal matter, often involving the use of water (often in the form of steam), alcohol, and/or other solvents.

For example, Korean Patent Registration No. 1018724910000 B1 describes an extraction apparatus that can be characterized as distillation extraction, which uses e.g. vacuum circulation in order to provide what it describes as enhanced extraction efficiency.

### SUMMARY

The following introduction is provided to introduce the reader to the more detailed discussion to follow. The introduction is not intended to limit or define any claimed or as yet unclaimed invention. One or more inventions may reside in any combination or sub-combination of the elements or process steps disclosed in any part of this document including its claims and figures.

Methods and apparatus disclosed herein may include the use of ultrasonic pre-treatment of a plant or fungal material feedstock. Such pre-treatment may have one or more advantages. For example, pre-treating the plant or fungal material feedstock with a high-frequency ultrasonic treatment was observed to increase the extraction efficiency for vacuum circulation extraction systems, such as those disclosed in Korean Patent Registration No. 1018724910000 B1.

It is believed that the extraction pressure in some vacuum circulation extraction systems was not high enough to completely rupture all of the cell walls of plant material subjected to extraction, particularly for plants with relatively thick cell walls.

In accordance with one broad aspect of this disclosure, there is provided a method for extracting one or more compounds from a plant or fungal material feedstock, the method comprising: immersing the plant or fungal material feedstock in a liquid; subjecting at least one of the immersed plant or fungal material and the liquid to ultrasonic vibrations; after the subjecting, extracting one or more compounds from the plant or fungal material.

In some embodiments, the liquid comprises at least one of water, ethanol, methanol, organic food-grade oils or a combination thereof.

In some embodiments, the ultrasonic vibrations have a frequency of between about 16 to 100 kHz. In some embodiments, the ultrasonic vibrations have a frequency of between 26 to 30 kHz.

In some embodiments, the ultrasonic vibrations are generated using a sonotrode.

In some embodiments, the extracting is conducted at a pressure of between about 0.10 MPa to about 0.2 MPa.

In some embodiments, the extracting is conducted at a temperature of between about 40° C. to about 150° C.

In some embodiments, the method further comprises, prior to the immersing, comminuting the plant or fungal material feedstock.

In some embodiments, comminuting the plant or fungal material comprises reducing an average particle size of the plant or fungal material to between 4.00 mm and 0.025 mm.

In some embodiments, comminuting the plant or fungal material comprises reducing an average particle size of the plant or fungal material to about 0.85 mm.

In some embodiments, the immersing and the subjecting are performed in the absence of chemical stabilizers.

Methods and apparatus disclosed herein may also include the use of ultrasonic post-treatment of compounds extracted from a plant or fungal material feedstock. Such post-treatment may have one or more advantages. For example, some plant extracts were observed to separate (e.g. come out of solution, or otherwise degrade) after a certain time following extraction. While post-extraction separation can be addressed by adding one or more chemical surfactants, this may not be considered desirable, particularly in the health and wellness industry.

Subjecting extracted compounds to a post-extraction ultrasonic treatment was observed to delay and/or reduce separation. This may advantageously provide extracts with a longer ‘shelf-life’. Also, stabilization without the use of chemical surfactants may be considered advantageous, even if the stabilized extracts do not have a materially longer shelf life than those with chemical stabilization.

In accordance with another broad aspect of this disclosure, there is provided a method for treating one or more compounds extracted from a plant or fungal material feedstock, the method comprising: providing the extracted one or more compounds and at least one liquid to an emulsification vessel; subjecting the extracted one or more compounds and the at least one liquid to ultrasonic vibrations using an agitator to create an emulsion of the extracted one or more compounds and the at least one liquid; and during the subjecting, circulating the agitator within the emulsification vessel.

In some embodiments, the at least one liquid comprises at least one of water, ethanol, methanol, organic food-grade oils or a combination thereof.

In some embodiments, the ultrasonic vibrations have a frequency of between about 16 to 100 kHz. In some embodiments, the ultrasonic vibrations have a frequency of between about 45 to 47 kHz.

In some embodiments, the agitator comprises a sonotrode.

In some embodiments, the extracted one or more compounds and the at least one liquid are subjected to ultrasonic vibrations until the emulsion has a polydispersity index of not more than 0.7. In some embodiments, the extracted one or more compounds and the at least one liquid are subjected to ultrasonic vibrations until the emulsion has a polydispersity index of not more than 0.3.

In some embodiments, the providing and the subjecting are performed in the absence of chemical stabilizers.

In accordance with another broad aspect of this disclosure, there is provided a method for extracting and stabilizing one or more compounds from a plant or fungal material feedstock, the method comprising: immersing the plant or fungal material feedstock in a liquid; subjecting at least one of the immersed plant or fungal material and the liquid to first ultrasonic vibrations; after the subjecting, extracting one or more compounds from the plant or fungal material; providing the extracted one or more compounds to an emulsification vessel; subjecting the extracted one or more compounds and at least one of the liquid and a post-extraction liquid to second ultrasonic vibrations using an agitator to create an emulsion containing the extracted one or more compounds.

In some embodiments, the method further comprises, during the subjecting, circulating the agitator within the emulsification vessel.

In some embodiments, the liquid comprises at least one of water, ethanol, methanol, organic food-grade oils or a combination thereof.

In some embodiments, the post-extraction liquid comprises at least one of water, ethanol, methanol, organic food-grade oils or a combination thereof.

In some embodiments, the first ultrasonic vibrations have a frequency of between about 16 to 100 kHz, optionally between about 26 to 30 kHz.

In some embodiments, the first ultrasonic vibrations are generated using a sonotrode.

In some embodiments, the second ultrasonic vibrations have a frequency of between about 16 to 100 kHz, optionally between about 45 to 47 kHz.

In some embodiments, the extracting is conducted at a pressure of between about 0.10 MPa to about 0.2 MPa.

In some embodiments, the extracting is conducted at a temperature of between about 40° C. to about 150° C.

In some embodiments, the extracted one or more compounds and the at least one of the liquid and a post-extraction liquid are subjected to ultrasonic vibrations until the emulsion containing the extracted one or more compounds has a polydispersity index of not more than 0.7, optionally not more than 0.3.

In some embodiments, the method further comprises, prior to the immersing, comminuting the plant or fungal material feedstock.

In some embodiments, comminuting the plant or fungal material comprises reducing an average particle size of the plant or fungal material to between 4.00 mm and 0.025 mm.

In some embodiments, comminuting the plant or fungal material comprises reducing an average particle size of the plant or fungal material to about 0.85 mm.

In some embodiments, the method is performed in the absence of chemical stabilizers.

It will be appreciated by a person skilled in the art that a method or apparatus disclosed herein may embody any one or more of the features contained herein and that the features may be used in any particular combination or sub-combination.

These and other aspects and features of various embodiments will be described in greater detail below.

### BRIEF DESCRIPTION OF THE DRAWINGS

For a better understanding of the described embodiments and to show more clearly how they may be carried into effect, reference will now be made, by way of example, to the accompanying drawings in which:

FIG. 1 is a simplified process flow diagram for a method for extracting one or more compounds from a plant material feedstock in accordance with one embodiment;

FIG. 2 is a schematic diagram of an apparatus for ultrasonic pre-treatment of a plant material feedstock in accordance with one embodiment;

FIG. 3 is a photograph of a sample produced using a distillation extraction system from a plant material feedstock that had been subjected to ultrasonic pretreatment, and a sample produced from a plant material feedstock that had not been subjected to ultrasonic pretreatment;

FIG. 4 is a data plot from a liquid chromatography-mass spectrometry (LC-MS) analysis in a positive mode, comparing extraction profiles of samples produced using a distillation extraction system;

FIG. 5 is a data plot from a LC-MS analysis in a negative mode, comparing extraction profiles of samples produced using a distillation extraction system;

FIG. 6 is a comparison of extraction profiles of FIG. 4;

FIG. 7 is a simplified process flow diagram for a method for extracting and stabilizing one or more compounds from a plant material feedstock in accordance with one embodiment;

FIG. 8 is a simplified process flow diagram for a method for extracting and stabilizing one or more compounds from a plant material feedstock in accordance with another embodiment; and

FIG. 9 is a data plot comparing the size distribution by intensity for an average of three untreated samples, and an average of three treated samples.

The drawings included herewith are for illustrating various examples of articles, methods, and apparatuses of the teaching of the present specification and are not intended to limit the scope of what is taught in any way.

### DESCRIPTION OF EXAMPLE EMBODIMENTS

Various apparatuses, methods and compositions are described below to provide an example of an embodiment of each claimed invention. No embodiment described below limits any claimed invention and any claimed invention may cover apparatuses and methods that differ from those described below. The claimed inventions are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed invention. Any invention disclosed in an apparatus, method or composition described below that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

The flowing is a description of a method for extraction which may be used by itself or in combination with one or more of the other features disclosed herein including the use of any of the features of the apparatus and/or and any of the methods disclosed herein.

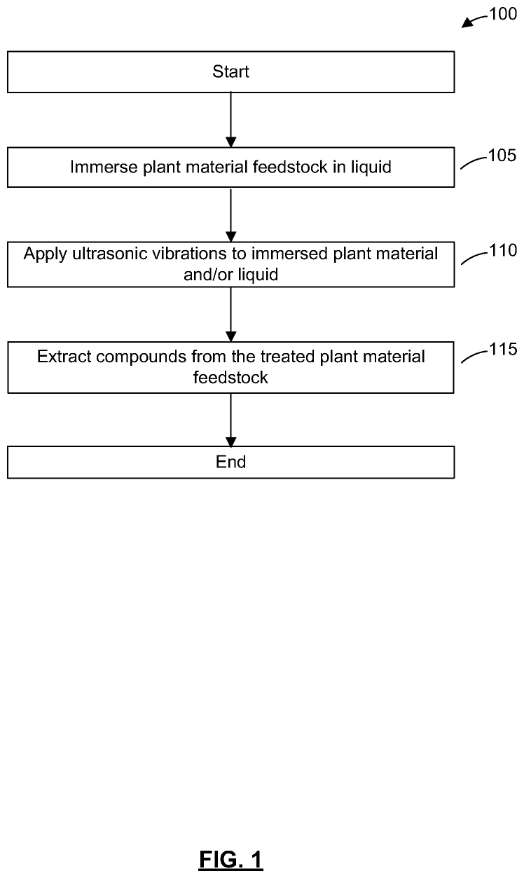

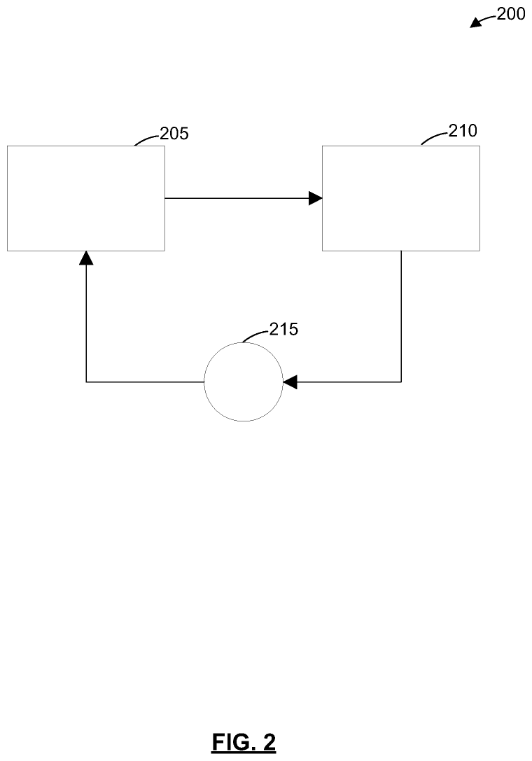

Referring to FIG. 1, there is illustrated a method 100 for extracting one or more compounds from a feedstock using an ultrasonic pre-treatment. The feedstock may be a plant material or a fungal material. Method 100 may be performed using the apparatus of FIG. 2, or any other suitable apparatus. In some embodiments, the feedstock may include cannabis, mushrooms, or other plant or fungal matter from which compounds may be extracted. The feedstock may be dried, fresh, or a combination thereof.

It will be appreciated that in the methods described below, the feedstock of plant material may alternatively be a feedstock of fungal material.

At 105, a feedstock of plant material is immersed in a liquid. For example, the plant material (e.g. cannabis) may be introduced into a vessel prior to, concurrently with, or following the introduction of the liquid to the vessel. With reference to FIG. 2, the plant material (e.g. cannabis) may be introduced into suspension chamber 205, along with the liquid in which it is immersed (e.g. water).

Optionally, prior to immersion in liquid, the feedstock of plant material may be ground or otherwise comminuted. For example, a plant material feedstock may be comminuted to have a particle diameter size of between 4.00 mm and 0.025 mm (e.g. so that particles pass through a sieve with a standard mesh size of between 5 and 500). Alternatively, the plant material may include substantially whole portions of the shoot system (e.g. leaves, stems, buds, fruits, etc.).

At 110, the immersed plant material and/or the liquid are subjected to ultrasonic vibrations. For example, a sonotrode may be placed into contact with the vessel. Additionally, or alternatively, a sonotrode may be at least partially immersed in the liquid. For example, a sonotrode may be circulated within the vessel.

In the example illustrated in FIG. 2, the immersed plant material and the liquid in which it is immersed may be circulated between suspension chamber 205 and an ultrasonic pretreatment chamber 210 (which contains a sonatrode) via a pump 215.

Preferably, the ultrasonic vibrations have a frequency of between about 16 to 100 kHz. More preferably, the ultrasonic vibrations have a frequency of about 26 to 30 kHz

Once the plant material has been immersed, process conditions within the vessel (pressure, temperature, vibration frequency, vibration intensity, etc.) may be controlled so that the plant material is sufficiently pre-treated.

It is believed that the application of ultrasonic vibrations may cause or promote the breakdown of cell walls within the plant material feedstock.

At 115, the treated plant material is subjected to an extraction process to extract one or more compounds. For example, the treated plant material may be subjected to a process that extracts compounds under relatively low temperatures (e.g. between about 40° C. to about 150° C.) and relatively low pressures (e.g. between about 0.1 MPa to about 0.2 MPa.

The extraction process may be performed using any suitable apparatus for the extraction of compounds from plant material. Preferably, the treated plant material is introduced to a distillation extraction system, such as the system disclosed in Korean Patent Registration No. 1018724910000 B1.

Preferably, method 100 is performed without the use of added chemical stabilizers.

Experimental testing has shown that the extraction yields using a distillation extraction system were materially improved when the plant material feedstock was subjected to ultrasonic pre-treatment.

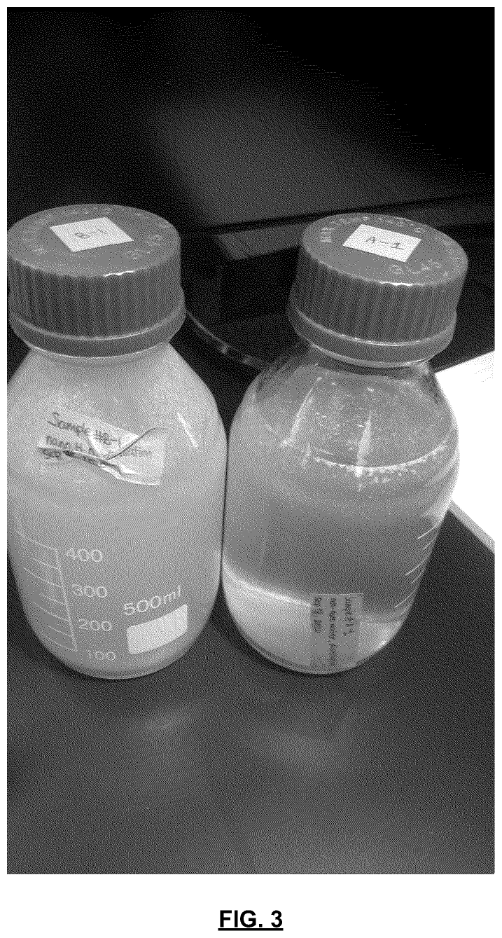

For example, FIG. 3 is a photograph comparing two samples produced using a distillation extraction system. The sample shown at left was produced from a cannabis feedstock that had been ground to an average particle size of about 0.85 mm (e.g. standard mesh size 20) that had been subjected to ultrasonic pretreatment using the apparatus of FIG. 2. Specifically, a cannabis feedstock immersed in water was circulated for about 1.5 hours at a flow rate of about 15 L/hr between a suspension chamber and an ultrasonic pretreatment chamber containing a sonatrode emitting ultrasonic vibrations of between about 26 to 30 KHz. The sample shown at right was produced from a ground cannabis feedstock that had not been subjected to ultrasonic pretreatment. Prior to the photograph of FIG. 3 being taken, the samples were refrigerated to promote the formation of precipitates.

As can be seen, the sample produced following ultrasonic pretreatment was cloudier than the sample produced with no ultrasonic pretreatment. It is thought that the increased cloudiness is indicative of an increase in non-water-soluble compounds, and thus indicative of a greater extraction efficiency resulting from the ultrasonic pretreatment.

Also, the extraction samples were subjected to liquid chromatography-mass spectrometry (LC-MS) analysis to compare a profile of cannabis extracts produced following ultrasonic pretreatment with a profile of cannabis extracts produced without ultrasonic pretreatment. To prepare the samples for LC-MS, 800 μL of sample was diluted in 400 μL of isopropanol, and subjected to vortex and sonication for 15 min. The clear solution was centrifuged before injection into the LC/MS apparatus. The LC-MS/MS method used an Agilent Poroshell 120 EC-C18 column (2.1×75 mm, 2.7 μm), and was 65 min, 0.3 mL/min with a gradient of:

| Time (min) | Pump B (%) |
|---|---|
| 0 | 5 |
| 45 | 95 |
| 55 | 95 |
| 55.1 | 5 |
| 65 | 5 |

The LC-MS/MS method was performed at 40° C., with A: H2O, 0.1% FA; B: ACN, 0.1% FA; Injection: 10 μL; and MS and MS/MS in positive and negative mode.

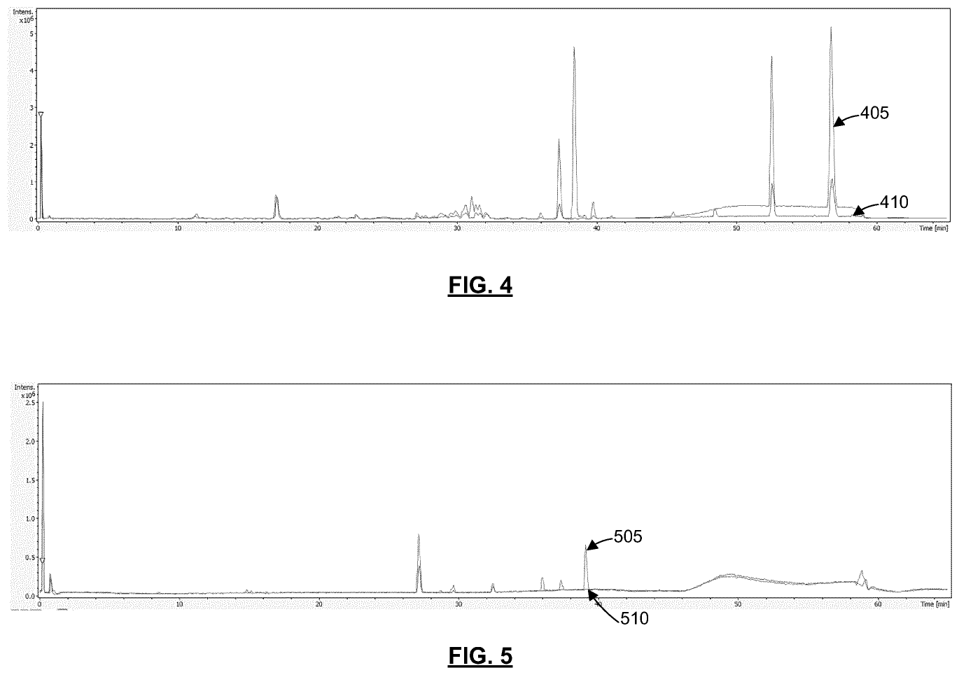

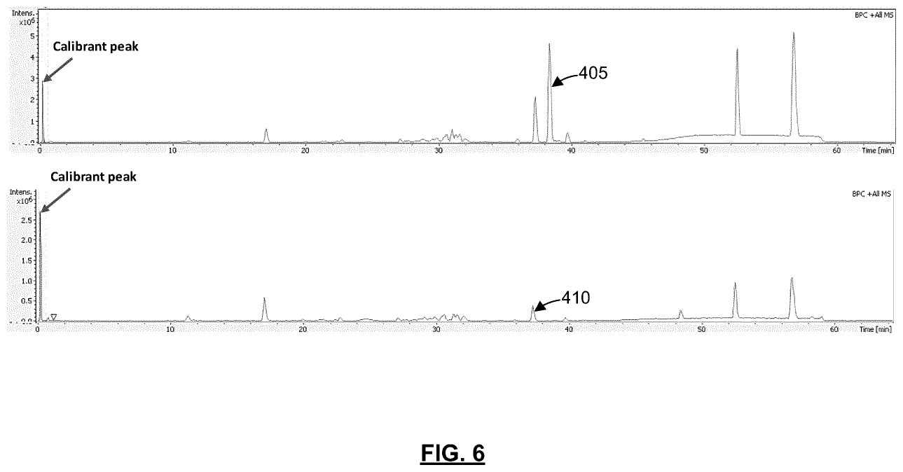

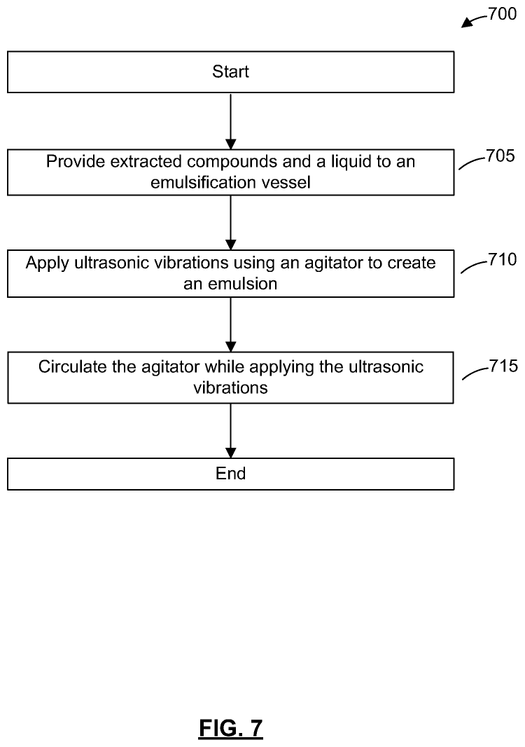

FIG. 4 is a data plot for a positive LC/MS mode, comparing the extraction profile 405 of the sample produced following ultrasonic pretreatment and the extraction profile 410 of the sample produced without ultrasonic pretreatment. In general, the extraction profile 405 has more and higher peaks, indicating a greater number and/or volume of extracted compounds as compared to the extraction profile 410. FIG. 6 illustrates the extraction profiles 405 and 410 separately.

FIG. 5 is a data plot for a negative LC/MS mode, comparing the extraction profile 505 of the sample produced following ultrasonic pretreatment and the extraction profile 510 of the sample produced without ultrasonic pretreatment. Consistent with FIG. 4, in general the extraction profile 505 has more and higher peaks, indicating a greater number and/or volume of extracted compounds as compared to the extraction profile 510.

Referring to FIG. 7, there is illustrated a method 700 for extracting and stabilizing one or more compounds from a plant material feedstock using ultrasonic stabilization. Method 700 may be performed using any suitable apparatus.

At 705, one or more compounds extracted from a feedstock of plant material and a liquid are introduced into an emulsification vessel. For example, output from a distillation extraction system, such as the system disclosed in Korean Patent Registration No. 1018724910000 B1, may be introduced into a vessel. In such situations, where the output contains both one or more extracted compounds and a liquid solvent (e.g. water), it may not be necessary to add an additional liquid.

At 710, the emulsification vessel and/or its contents are subjected to ultrasonic vibrations. For example, an agitator (e.g. a sonotrode) may be placed in contact with or within the vessel. Preferably, the ultrasonic vibrations are continuously applied to prevent coalescence, creaming, and/or flocculation.

Preferably, the ultrasonic vibrations have a frequency of between 16 kHz to 100 kHz.

At 715, the agitator is continuously circulated within the vessel.

During the application of the ultrasonic vibrations, process conditions within the emulsification vessel (pressure, temperature, vibration frequency, vibration intensity, etc.) may be controlled to promote the formation of a consistent emulsion.

Preferably, method 700 is performed without the use of added chemical stabilizers.

It is believed that the application of ultrasonic vibrations may cause or promote the formation of a relatively stable emulsion. In this respect, experiments were conducted using a particle size analyzer to assess particle size within untreated output from a distillation extraction system, and within output from a distillation extraction system after post-extraction ultrasonic treatment.

For example, in one set of experiments, samples of commercially available cannabis oil and water were blended using a commercial blender for about one minute. A first set of samples were then processed according to method 700, with ultrasonic vibrations of between 45 to 47 kHz being applied for about one hour, and then blended again using the commercial blender for about one minute. A second set of samples were rested for about one hour, and then blended again using the commercial blender for about one minute. For both sets of samples, milky-colored emulsions were formed.

As summarized in Table 1 below, the second set of samples showed an average particle size (diameter) for CBD molecules of 116.1 nm, and an average particle size (diameter) for THC molecules of 22.85 nm. The first set of samples (processed according to method 700 as noted above) showed an average particle size (diameter) for CBD molecules of 108.4 nm, and an average particle size (diameter) for THC molecules of 16.49 nm. Thus, the average particle of CBD molecules reduced 7.4% in size, and the average particle of THC molecules reduced 44.3% in size.

Another measurement for emulsions is a polydispersity index (PdI), which measures the heterogeneity of a sample based on size. PdI is commonly used in pharmaceutics to test for stability of a product. Larger PdI values, close to 1, means there is high size variations among substances, which will readily separate to hydrophobic and hydrophilic layers. Lower PdI values, close to 0, means the particles are similar in size, which indicates a more stable emulsion.

Test results indicated a lower polydispersity index (PdI) in samples subjected to post-extraction ultrasonic treatment. Representative results are shown below in Table 1:

| | | Average CBD particle | Average THC particle |
|---|---|---|---|
| | PdI | size (d · nm) | size (d · nm) |
| Untreated sample | 0.401 | 116.1 | 23.85 |
| Treated sample | 0.282 | 108.4 | 16.49 |

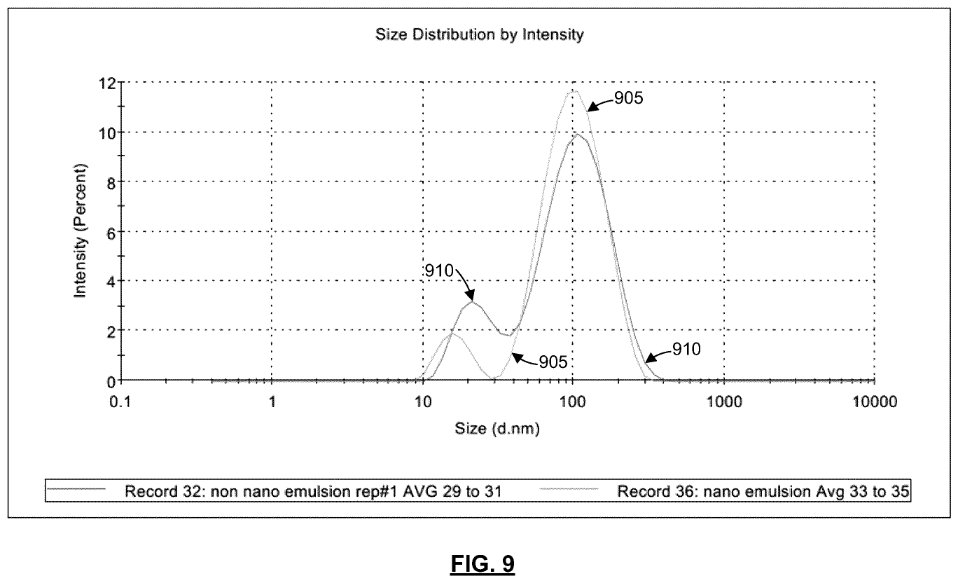

FIG. 9 is a data plot comparing the size distribution by intensity for an average of three untreated samples, and an average of three treated samples.

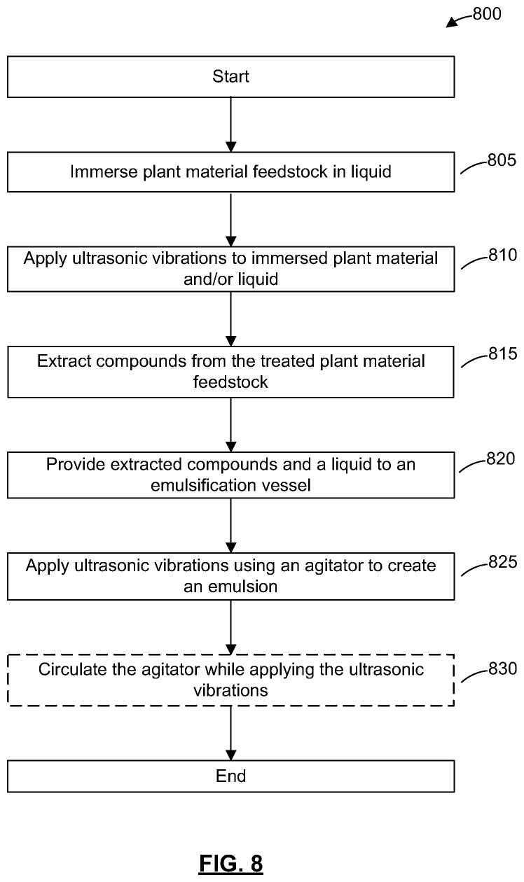

Referring to FIG. 8, there is illustrated a method 800 for extracting and stabilizing one or more compounds from a plant material feedstock using ultrasonic pre-treatment and ultrasonic post-extraction stabilization. Method 800 may be performed using any suitable apparatus.

Method 800 includes an ultrasonic pre-treatment of a plant material feedstock, and a post-extraction ultrasonic stabilization treatment. Performing both feedstock pre-treatment and extract post-treatment may result in an overall process with a high extraction efficiency and a relatively shelf-stable plant extract, and can preferably be performed without the use of added chemical stabilizers.

At 805, a feedstock of plant material is immersed in a liquid, e.g. as discussed above for 105 in method 100.

Optionally, prior to immersion in liquid, the feedstock of plant material may be ground or otherwise comminuted.

At 810, the immersed plant material and/or the liquid are subjected to ultrasonic vibrations, e.g. as discussed above for 110 in method 100.

At 815, the treated plant material is subjected to an extraction process to extract one or more compounds, e.g. as discussed above for 115 in method 100.

At 820, the extracted one or more compounds are introduced into an emulsification vessel, with an optionally added liquid, e.g. as discussed above for 705 in method 700.

At 825, the emulsification vessel and/or its contents are subjected to ultrasonic vibrations using an agitator, e.g. as discussed above for 710 in method 700.

At 830, optionally the agitator is continuously circulated within the vessel, e.g. as discussed above for 715 in method 700.

Preferably, method 800 is performed without the use of added chemical stabilizers.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

It should be noted that terms of degree such as “substantially”, “about” and “approximately” as used herein mean a reasonable amount of deviation of the modified term such that the end result is not significantly changed. These terms of degree may also be construed as including a deviation of the modified term, such as by 1%, 2%, 5% or 10%, for example, if this deviation does not negate the meaning of the term it modifies.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

1.-19. (canceled)

20. A method for extracting and stabilizing a compound from a plant or fungal material feedstock, the method comprising:
immersing the plant or fungal material feedstock in a liquid;
subjecting the immersed plant or fungal material and the liquid to a first ultrasonic vibration treatments, to obtain a first ultrasonicated sample;
subjecting the first ultrasonicated sample to a distillation for extracting the compound from the plant or fungal material in a distilled sample;
subjecting the distilled sample comprising the extracted compound and to a second ultrasonic vibrations treatment using an agitator to create an emulsion containing the extracted compound.

21. The method of claim 20, further comprising circulating the agitator within an emulsification vessel during the second ultrasonic vibration treatment.

22. The method of claim 20, wherein the liquid comprises at least one of water, ethanol, methanol, organic food-grade oils or a combination thereof.

23. The method of claim 20, wherein the post-extraction liquid comprises at least one of water, ethanol, methanol, organic food-grade oils or a combination thereof.

24. The method of claim 20, wherein the first ultrasonic vibration has a frequency of between about 16 to 100 kHz.

25. The method of claim 20, wherein the first ultrasonic vibration obtained from a sonotrode.

26. The method of claim 20, wherein the second ultrasonic vibration has a frequency of between about 16 to 100 kHz.

27. The method of claim 20, wherein the distillation is conducted at a pressure of between about 0.10 MPa to about 0.2 MPa.

28. The method of claim 20, wherein the distillation is at a temperature of between about 40° C. to about 150° C.

29. The method of claim 20, wherein the distilled sample comprising the extracted is subjected to the second ultrasonic vibration until the emulsion reaches a polydispersity index of not more than 0.7.

30. The method of claim 20, further comprising comminuting the plant or fungal material feedstock prior to the step of immersing.

31. The method of claim 30, wherein comminuting the plant or fungal material comprises reducing an average particle size of the plant or fungal material to between 4.00 mm and 0.025 mm.

32. The method of claim 31, wherein comminuting the plant or fungal material comprises reducing an average particle size of the plant or fungal material to about 0.85 mm.

33. The method of claim 20, wherein the method is performed in the absence of chemical stabilizers.

34. The method of claim 24, wherein the first ultrasonic vibration has a frequency of between about 26 to 30 kHz.

35. The method of claim 26, wherein the second ultrasonic vibration has a frequency of between about 45 to 47 kHz.

36. The method of claim 29, wherein the polydispersity index is of not more than 0.3.
